(19) United States
(12) Patent Application Publication
(10) Pub. No.: US 2024/0306891 A1
(43) Pub. Date: Sep. 19, 2024

(54) MEDICAL DEVICE WITH TURBIDITY MEASUREMENT SENSOR

(71) Applicant: GYRUS ACMI, INC D/B/A OLYMPUS SURGICAL TECHNOLOGIES AMERICA, Westborough, MA (US)

(72) Inventors: David M. Leak, Brooklyn Park, MN (US); Kester Julian Batchelor, Mound, MN (US); William E. Marvin, III, Attleboro, MA (US); Kimbolt Young, Newtonville, MA (US)

(21) Appl. No.: 18/595,956

(22) Filed: Mar. 5, 2024

Related U.S. Application Data
(60) Provisional application No. 63/490,127, filed on Mar. 14, 2023.
(60) Provisional application No. 63/582,621, filed on Sep. 14, 2023.

(51) Int. Cl. A61B 1/00; A61B 1/015; A61B 1/018; A61B 1/06; A61B 17/00; A61B 17/22; A61B 18/00; A61B 18/26; A61B 34/00; G16H 40/63

(52) U.S. Cl. CPC A61B 1/00097; A61B 1/00006; A61B 1/018; A61B 17/22012; A61B 18/26; A61B 34/25; G16H 40/63; A61B 1/015; A61B 1/0655; A61B 2017/0034; A61B 2018/00511; A61B 2018/00577; A61B 2018/00982

(57) ABSTRACT

A system can automatically regulate irrigation, suction, or both, such as during a lithotripsy procedure. The system can include one or more sensors. Processing circuitry can be configured to generate an indication of a level of turbidity of a solution at or near a distal tip of a flexible scope, such as based on one or more signals from the one or more sensors. A control signal can be used to adjust an irrigation flow rate and/or a suction rate at the distal tip of the flexible scope. The level of turbidity can be determined using a sensor to measure electrolytic conductivity of the solution, a sensor to measure electromagnetic induction across a coil at the distal tip of the flexible scope and/or a measure of back reflected light or a measure of absorbed light emitted into the solution by a light source and measured by a light sensor.

### PRIORITY CLAIM

This application claims the benefit of priority to U.S. Provisional Patent Application Ser. No. 63/490,127, filed Mar. 14, 2023, and U.S. Provisional Patent Application Ser. No. 63/582,621, filed Sep. 14, 2023, the contents of which are incorporated herein by reference.

### TECHNICAL FIELD

The present disclosure relates a system to provide a measure of surgical scene clarity such as while treating a calculus such as a kidney stone during lithotripsy.

### BACKGROUND

Treating a calculus such as a kidney stone via lithotripsy is a surgical procedure involving in-situ observation of the surgical scene within a patient near the target. The observation can use an imaging device such as a camera attached to a scope, such as a ureteroscope, an endoscope, or another similar device. Lithotripsy directs energy, such as ultrasonic or laser energy toward the target stone(s) to break the target into smaller pieces that can subsequently be removed by an irrigation system.

Treating kidney stones creates suspended solids in solution. This can impair a physician's visibility of the surgical scene when viewed through the scope. Thus, the visual clarity of the surgical scene is based at least in part on the degree of suspended solids in the solution.

### SUMMARY

Disclosed herein are systems and methods for assessing the clarity of the surgical scene, such as can be based at least in part on the degree of suspended solids in a solution of a surgical site (e.g., at a distal tip of a scope used in a lithotripsy procedure). For example, a system can provide a measure of surgical scene clarity, which, in turn can be used to provide automatic feedback to the irrigation system of the scope. A system may use a flexible scope containing illumination fibers and an imaging system capable of use for observing the surgical scene. Such a scope can be equipped with one or more working channels, including at or near the distal tip of the scope, such as can be used to provide functionality such as irrigation, suction, grasping, or the like.

During lithotripsy, a stone, such as a kidney stone, can be broken into smaller pieces, such as by applying electromagnetic or acoustic energy to the target stone. These smaller pieces of debris can become suspended in a solution, such as at or near the surgical scene, and then evacuated by one or both of an irrigation or suction system. Scene visibility can be impacted or affected by the degree of breakdown, the size, and/or the number of suspended solids emanating from the stones as they are broken. Disclosed herein is a system to detect and quantify a measure of relative clarity (e.g., a degree of cloudiness) of the solution, referred to as turbidity, such as at or near the surgical scene. The system can measure the turbidity using a sensor included on or in a medical device. Based on the degree of turbidity, a system can provide one or more of an instruction, a control signal, or feedback to the connected irrigation system. An instruction can be to adjust a setting of the irrigation system, either automatically, or by instructing a user to do so or by giving a user an opportunity to confirm whether to carry out the recommended instruction. For example, the irrigation system can be instructed to adjust the irrigation flow rate so that the amount of irrigation solution, such as saline, is increased, decreased, or stopped. In another example, the system can provide multiple instructions or control signals. For example, the system can cause irrigation to stop or cease or to be reduced and simultaneously (or substantially simultaneously) cause suction to be initiated, started, or increased, such as in response to a detected turbidity exceeding a threshold value or meeting another specified criterion. In another example, the system can cause both irrigation and suction to increase together, such as in response to a detected turbidity exceeding a threshold value or meeting another specified criterion.

Thus, a medical system capable of determining a turbidity level and/or surgical scene clarity can cause the irrigation system of a device to adjust the flow rate of irrigation solution and/or the suction rate at the surgical scene (e.g., at the distal tip of a flexible scope). The determined turbidity level can correspond to a measure of how clear the medium at the surgical scene is and quantitate that to a value within a range of possible values. The quantitated value can then be passed or relayed to processor circuitry, such as for comparison to a threshold value or one or more other criteria. The resulting information can be used to generate a feedback or other control signal, such as can be communicated to an analog sensor or a pump (or pump control), which can be used to control an amount of irrigation and/or suction or aspiration at the distal tip of a scope. A measurement of high turbidity or low clarity can result in a signal being sent to the irrigation system to increase the flow of irrigation fluid and/or adjust the corresponding rate of suction. A measurement of low turbidity or high clarity can result in a signal being sent to the irrigation system to decrease the flow of irrigation and/or adjust a corresponding rate of suction.

In an example, the medical system can automatically regulate irrigation and/or suction without physician intervention (e.g., automatically) based on the turbidity measurement. The automatic regulation can be performed by an artificial intelligence (AI) or machine learning (ML) or other algorithm (e.g., a non-AI or non-ML deterministic algorithm) or process. Additionally, or alternatively, the adjustment can be made using a hardware-based feedback loop or feedback control. In another example, the algorithm can output a recommendation to adjust a setting of the irrigation and/or suction to the physician, which the physician can accept or reject.

Additionally or alternatively, the medical system can determine turbidity levels at different times and determine that the surgical field is not clear enough (as the procedure progresses) and send a signal to the ablation source to lower or terminate the ablation energy. For example, as a stone is ablated and the turbidity level of the surgical scene or at the distal tip of the scope increases, the controller circuitry can cause a laser, ultrasonic transducer, thermal transducer, or the like, to stop emitting ablation energy until the scene is cleared. Additionally, or alternatively, the medical system can respond to increased turbidity by sending a signal to a light source or emitter to adjust the brightness of an aiming beam or other illumination light source.

Additionally or alternatively, the turbidity level can be used to determine or estimate how much time is required to finish the procedure. For example, a sensor, such as a particle counter, can be included in the medical system and can be used to determine how much material has been removed from the stone or target scene and/or a rate of ablation or how quickly material is being removed from the stone or target scene. Based at least in part on this determination, the system can provide an indication, such as on a graphical user interface (GUI), to the physician or other user. The indication can include how much time is estimated to finish ablation of the stone and/or how much time is estimated to finish the overall procedure. Such an indication or estimate can be especially helpful when the procedure includes ablating multiple stones. Additionally, or alternatively, the medical system can use the sensor data to determine a current turbidity level or percentage (e.g., how much dust, debris, particles, or the like is currently in the surgical field) and display that information on the GUI.

Other examples of indications that the medical system can output to a physician include a warning or error message or a recommendation. For example, the medical system can provide an indication when excess vibration is detected, such as via an accelerometer that can be located at or near a distal tip of the scope. The greater the turbidity level, the more vibration can occur at the tip of the scope, which can negatively impact the procedure. The medical system can also output a recommendation to the physician to move, adjust, reposition, or the like, the tip of the scope based on the turbidity level. For example, a recommendation can be made to move the tip of the scope to a portion of the surgical field with a lower amount of turbidity. In another example, instead of automatically adjusting a source of illumination or ablation energy as discussed above, the medical system may provide a recommendation to the physician to change a setting of the medical device to a new value, such as to allow user-adjustment or user-confirmation of adjustment. The new value can include an amount of ablation energy, a new brightness level or percentage of an illumination source, or the like.

Thus, a medical system can include one or more sensors and processing circuitry to measure or determine a level of turbidity of a solution at or near a distal tip of a flexible scope. The level of turbidity can be determined using one or more sensors of the medical system or coupled thereto. For example, a sensor can measure electromagnetic induction across a coil at the distal tip of the flexible scope to determine turbidity. In another example, the one or more sensors can include an imaging or optical sensor such as a camera to collect imaging information. In such an example, a field clarity level can be determined based at least in part on the imaging information captured by the imaging sensor (e.g., in conjunction with data collected from other sensors).

Another sensor can measure the rate at which material is breaking off of or being removed from the stone. In another example, light can be emitted into the solution by a light emitting diode (LED) and a turbidity level can be determined based on behavior of the light. For example, the one or more sensors can include a sensor to measure an amount of the emitted light that is back reflected toward an optical sensor included or connected to the scope (nephelometric turbidity). Additionally, or alternatively, the one or more sensors can include a sensor to measure or determine an amount of the emitted light that is absorbed by the solution (absorption turbidity).

The level of turbidity or scene clarity can be determined by the processing circuitry based at least in part on a correlation of the signal or signals from the one or more sensors to a level of turbidity (e.g., using a correlation function and/or a lookup table). Based on the correlation, the processing circuitry can provide a signal to an irrigation system of the medical device connected to the flexible scope. The signal can control or adjust at least one of: i) an irrigation flow rate and/or ii) a suction rate at the distal tip of the flexible scope.

A benefit of automatic adjustment of the irrigation and/or suction, such as based on scene clarity or turbidity, is that the automatic adjustment can result in a more efficient and productive stone removal procedure. A more efficient and productive stone removal procedure can result in shorter overall procedure times or lengths. Lithotripsy procedures are less efficient when the level of turbidity is not reduced. Therefore, a system that can automatically control irrigation or suction to reduce turbidity can allow the physician to remain focused on the task of removing the stone while the system automatically maintains the surgical scene clarity. Another benefit of the present system is that since debris is removed more efficiently, more debris can be removed during the procedure. Thus, the need for future or follow-up procedures can be reduced or lowered. Also, the data from the sensors and the corresponding turbidity measurements throughout the procedure can be used to determine an overall efficiency level or score for the procedure. This efficiency score can in turn be used in subsequent analysis of the procedure, effectiveness or efficiency of the physician or surgical team, or the like.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a medical device which can include a turbidity sensor.

FIG. 2 illustrates an example of a turbidity sensor using electrolytic conductivity which can be included in a medical device such as the medical device of FIG. 1.

FIG. 3 illustrates an example of a turbidity sensor using electromagnetic induction which can be included in a medical device such as the medical device of FIG. 1.

FIG. 4 illustrates an example of a nephelometric turbidity sensor which can be included in a medical device such as the medical device of FIG. 1.

FIG. 5 illustrates an example of an absorption turbidity sensor which can be included in a medical device such as the medical device of FIG. 1.

FIG. 6 illustrates an example of a cross-section of a fiber tip that can be included at the distal end of a flexible scope.

FIG. 7 illustrates an example of a method of determining at least one of turbidity or surgical scene clarity.

FIG. 8 is a block diagram of an example of an apparatus, device, or machine upon which any one or more of the techniques (e.g., methodologies) discussed herein may perform.

FIG. 9 shows a schematic diagram of an exemplary computer-based clinical decision support system (CDSS).

### DETAILED DESCRIPTION

Treatment of kidney stones can include breaking the stones into smaller pieces. The resulting smaller pieces can become suspended solids in solution. These suspended solids can then be evacuated using an irrigation and/or suction system. Scene visibility can be impacted by the degree of breakdown (e.g., the size and the number) of the suspended solids, which can impair a physician's visibility of the surgical scene when viewed through a scope. The present inventors have recognized, among other things, a need for an improved system for assessing the visual clarity of a surgical scene based on the degree or amount of suspended solids in the solution. The present inventors have also recognized a need for a system that makes removal of the suspended solids more efficient so that a physician can focus on stone ablation.

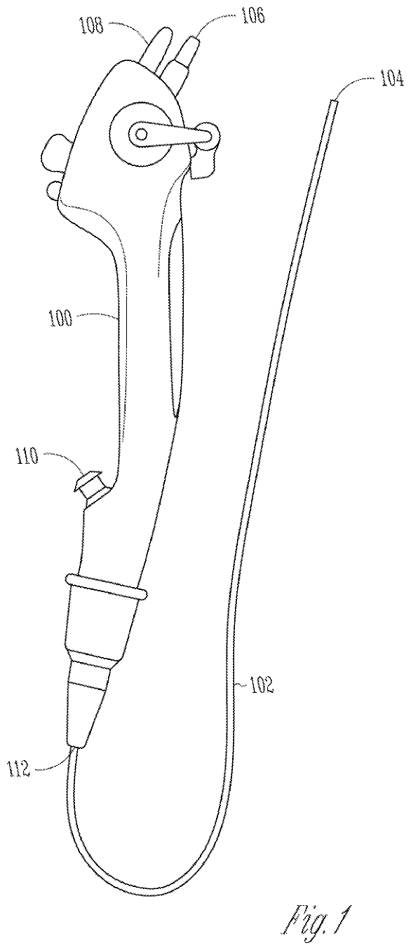

FIG. 1 illustrates an example of a medical device that can include a turbidity sensor. As illustrated in FIG. 1, the medical device can include a device body 100 or handle and a flexible scope 102 such as an endoscope connected to the device body 100 at a proximal end 112. The medical device can further include an irrigation port 106, an input-output (I/O) connection 108, and an accessory port 110. The flexible scope 102 (e.g., the flexible endoscope portion) can include a longitudinal lumen working channel in an interior portion of the flexible scope 102. The longitudinal working channel can span, run, or the like from the proximal end 112 to a distal end 104 or tip and can be used for irrigation and/or suction, can include a camera and/or a light source (e.g., as in a fiber or solid state), or the like.

A turbidity sensor can be placed internal to a lumen of the flexible scope 102 or external to the flexible scope 102 and/or the device body 100. For example, a turbidity sensor can be placed at one or more of: the distal end 104, the irrigation port 106, and/or at a location between the distal end 104 and the irrigation port 106 (such as an interior portion of the device body 100). That is, measurements by the one or more sensors disclosed (and discussed below) can be made or collected at different locations corresponding to the medical device. For example, a sensor located between the distal end 104 of the flexible scope 102 and the irrigation port 106 (e.g., a particle counter) can record the number or amount of floating elements or particles being evacuated. In another example, a particle counter located at or near the distal end 104 can record how many floating elements or particles are currently in the solution or medium, such as at the end of the ablation procedure. Then, a rate or amount of irrigation and/or suction can be determined (or adjusted) based on the number of particles counted by the particle counters.

A current particle count can help a physician ensure that all debris from the ablated stone can be removed before the procedure is completed. Leftover debris that is not removed can be difficult for the subject to pass naturally during urination, can act as potential “seeding elements” for growing new stones. Furthermore, data from multiple sensors can be used in conjunction with each other. For example, an imaging sensor can be used to identify potentially compromised tissue where a new stone may form, which can in turn be treated by the physician. Thus, the data from the particle counter and the imaging sensor can be used together to reduce the risk of new stone formation.

The device input and output circuitry can pass the one or more sensor signals to a system controller or other controller circuitry (operating the algorithms discussed herein) connected to the medical device at I/O connection 108. In lithotripsy procedures the sensor or sensors can be used in combination with diagnostic or therapeutic energy devices, such as ultrasound, ultrasonic, thermal, RF, or laser lithotripsy systems. For example, the amount of energy emitted from the lithotripsy energy devices can be controlled or adjusted, such as based on the turbidity level or surgical scene clarity determined from the sensor data.

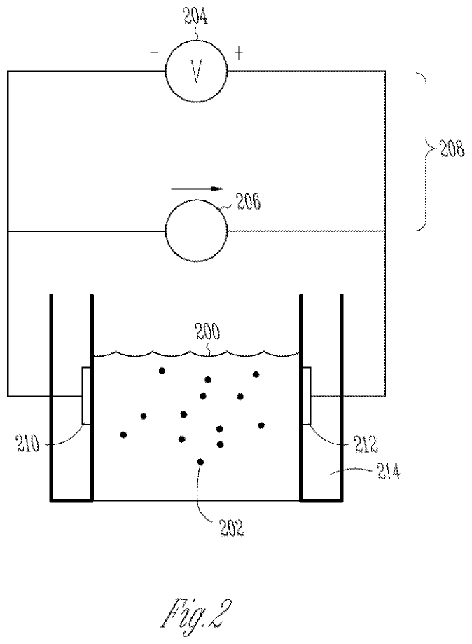

FIG. 2 illustrates an example of a turbidity sensor, using electrolytic conductivity to determine turbidity, which can be included in a medical device such as the medical device of FIG. 1. Electrical conductivity of the solution can be measured by the turbidity sensor, such as between two electrodes (210 and 212) separated by a fixed distance. The electrodes 210 and 212 can be included in or near a working channel 214 and/or an irrigation port or a distal tip of a flexible scope such as an endoscope. The degree of electrical conductivity of a solution 200 in which the distal tip of a scope is located can depend on the ion density (or particle density) of the solution 200. Lithotripsy procedures can produce ions 202 or particles suspended in the solution 200. As such, a conductance per centimeter of the solution 200 can be determined using a direct voltage measurement in response to a specified-value test or excitation current, or vice-versa. The voltage or current measurements can be taken by a voltage sensor 204 and/or a current sensor 206 located on a sensing circuit 208. The measured conductivity response signal can be regarded as a function of the electrode geometry and spacing of the two electrodes 210 and 212 and can be correlated with turbidity. The resulting measured conductivity response signal can be used as an index into a correlation function or look-up table, which can provide a measure of turbidity.

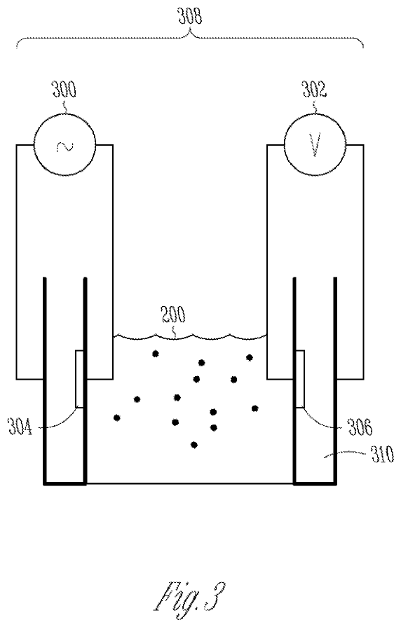

FIG. 3 illustrates an example of a turbidity sensor, using electromagnetic induction, which can be included in a medical device such as that of FIG. 1. Electromagnetic coupling can be created between an AC voltage source 300 and the fluid or solution 200. The electromagnetic coupling can be measured by an AC voltmeter 302. The AC voltage source 300 and the AC voltmeter 302 can be components included on a sensing circuit 308. A primary coil 304 can be energized by AC power and can induce an electric current that passes through the sample solution 200. This induced current, in turn, can induce a measurable voltage in a secondary coil 306, which can be measured by the AC voltmeter 302. The primary coil 304 and/or the secondary coil 306 can be located in a working channel 310 and/or an irrigation port of a flexible scope such as the flexible scope 102 discussed above.

A correlation between the measured voltage in the secondary coil 306 and turbidity can be determined, such as using a correlation function or a lookup-table to provide a measure of turbidity based on the measured voltage. The measure of turbidity can be a function of the geometry of the coils and the spacing between the coils. The AC voltage source 300 and/or the AC voltmeter 302 can be located in the working channel used by an optical fiber (or fiber bundle) that is inserted into the flexible scope 102. For example, the AC voltage source 300 and/or the AC voltmeter 302 can be located at the tip of the fiber that is inserted into the flexible scope 102, such as extending out of the flexible scope 102 through the distal tip 104 of the flexible scope 102, and/or can be located at the irrigation port 106 of the medical device.

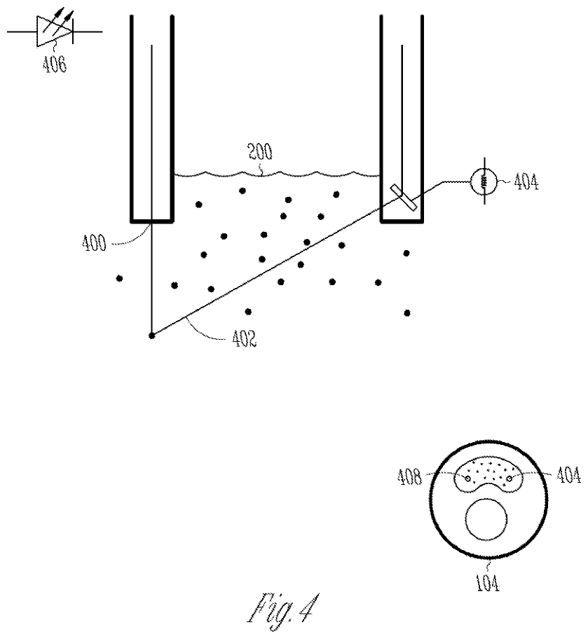

FIG. 4 illustrates an example of a nephelometric turbidity sensor that can be included in a medical device such as that of FIG. 1. A light source, such as a light emitting diode (LED) 406, may emit illumination light 400 into the solution 200. In response to the received illumination light, the solution 200 can provide scattered response light 402 that can be detected by a light sensor 404. The degree of response light back reflection (e.g., an amount of back-reflected light), in response to a given amount of illumination light, can be measured using the light sensor 404. The measured about of back-reflected light can be converted to an electrical signal. The electrical signal can provide an indication of turbidity in the solution 200, such as by using a correlation function, a look-up table, or the like.

The LED 406 and/or the light sensor 404 can be fiber based, such as by being included or integrated in an illumination fiber or fiber bundle that can be inserted into the flexible scope 102, such as illustrated in FIG. 4 showing the cross-section of the fiber tip 408 at the distal end 104 of the flexible scope 102.

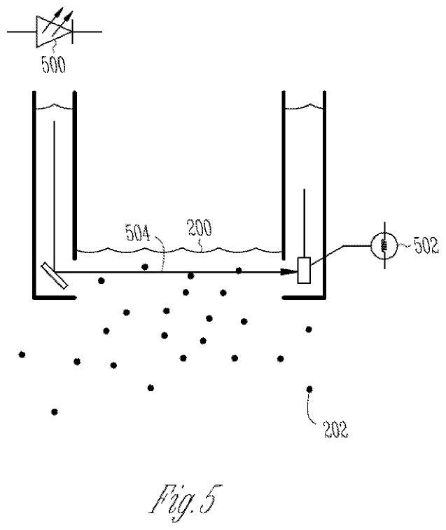

FIG. 5 illustrates an example of an absorption turbidity sensor that can be included in a medical device such as that of FIG. 1. In FIG. 5, a light source such as an LED 500 can be located opposite a light sensor 502. Between the LED 500 and the light sensor 502, suspended solids (the ions 202) in the solution 200 attenuate the emitted light 504 illuminating the solution 200. A detected portion of the emitted light 504 can be converted by the light sensor 502 into an electrical signal, which, in turn can be used to generate a turbidity value, such as via a correlation function, a look-up table, or the like. Thus, the degree of absorption of the illumination light by the solution 200 can provide a measure of turbidity. Such an arrangement can also be fiber based and included in or integrated into an illumination fiber or fiber bundle, as similarly discussed above.

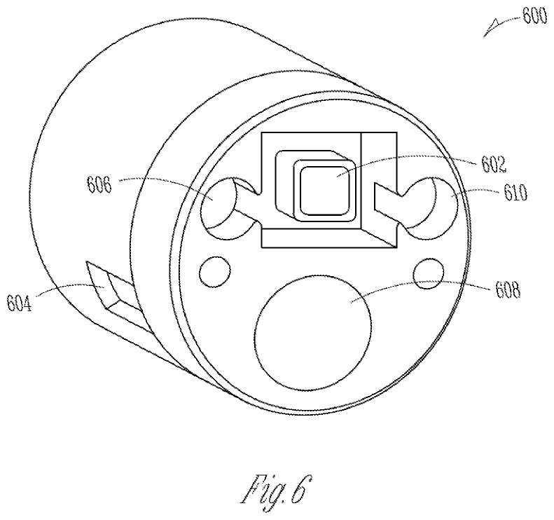

FIG. 6 illustrates an example of a cross-section of a fiber tip 600 that can be included at the distal end 104 of the flexible scope 102 illustrated in FIG. 1. In an example, the fiber tip 600 can include one or more sensors. The one or more sensors can include an optical sensor 602 (e.g., a camera) and/or an environmental sensor 604. The environmental sensor 604 can include a temperature sensor and/or a pressure sensor, or any other sensor capable of sensing or measuring a parameter of the environment or medium in which the fiber tip 600 is located. The one or more sensors can also include one or more of the turbidity sensors discussed above for FIGS. 2-5.

The fiber tip 600 can also include one or more channels such as a laser channel 606 into which a laser fiber or an optical fiber can be inserted or included and/or through which laser radiation or light (visible or invisible) can be emitted. The one or more channels can also include a suction channel 608 (a working channel). The suction channel 608 can be connected to a pump, a vacuum, or the like, to allow particles such as fragmented stone to be sucked out of or otherwise removed from the surgical field. The fiber tip 600 can also include an irrigation channel 610. The irrigation channel 610 can be connected to an irrigation system, a pump, or the like. Irrigation fluid such as saline can be pumped or emitted from the irrigation channel 610, to clear smaller, dust-like particles from the surgical field that are too small to be sucked through the suction channel 608.

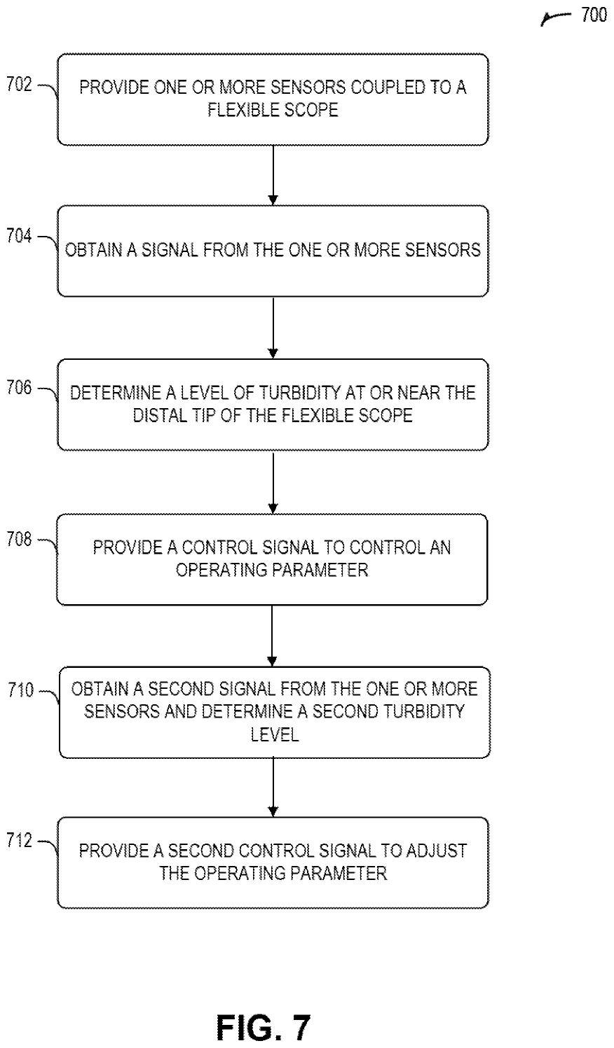

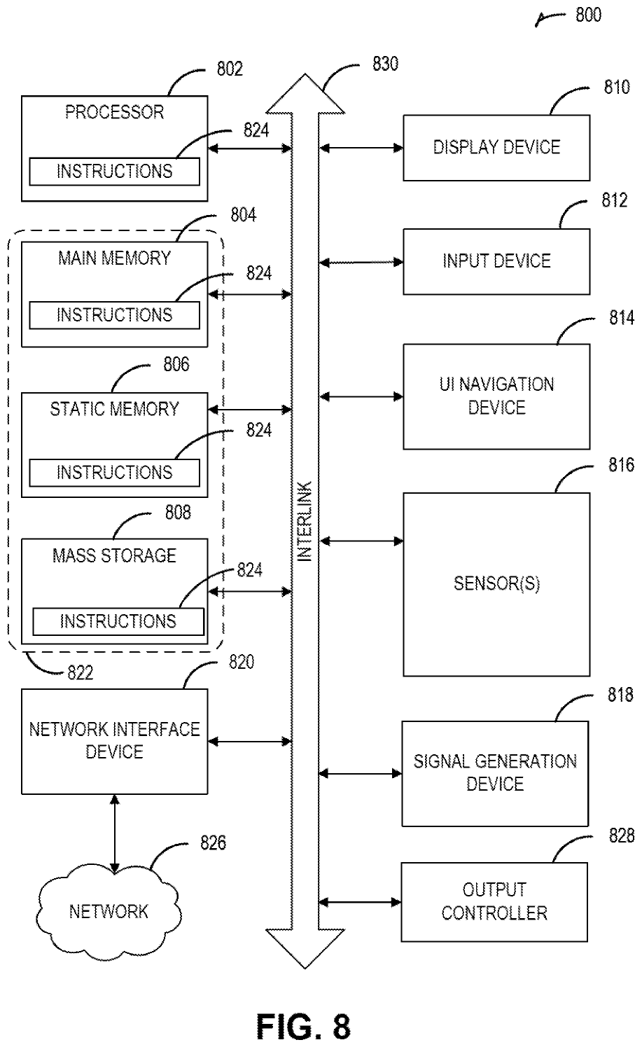

FIG. 7 illustrates an example of a method 700 of determining at least one of turbidity or surgical scene clarity. The method 700 can include or comprise a number of Operations or Steps (702-712). These Operations are exemplary, and the method can omit one or more of the listed Operations, can repeat Operations, can include other Operations, or can execute the Operations concurrently, substantially simultaneously, or in another order, as appropriate or desired. The operations can be performed automatically by the processor or controller of a machine or computer, such as described below for FIG. 8.

At 702, the method 700 can include providing one or more sensors coupled to a flexible scope. The flexible scope can be an endoscope or other similar scope connected to the handle or body of a medical device. The one or more sensors can include an environmental sensor, such as a temperature or pressure sensor, one or more particle counters, or one or more of the turbidity sensors discussed above for FIGS. 2-5. At 704 the method 700 can include obtaining a signal from the one or more sensors, and at 706 can include determining a level of turbidity of a solution at or near a distal tip of the flexible scope.

The solution can be a medium (e.g., air, water, saline, or the like) in which a target such as a kidney stone is located. Determining the level of turbidity can be based at least in part on a signal from the one or more sensors and at least one of a correlation function or a lookup-table. The correlation function and/or look-up table can correlate the signal from the one or more sensors to the level of turbidity and/or surgical scene clarity. The turbidity level can correspond to a level of cloudiness or a visibility level at the distal tip and can be based on how much of the target has been ablated.

At 708, the method 700 can include providing a signal to control at least one operating parameter (e.g., control an amount or flow of irrigation or suction at or near the distal tip of the flexible scope). For example, when the turbidity level is high (meaning the medium contains a high amount dust and/or particles from the ablated target and visibility of the surgical scene is low) irrigation fluid can be pumped from a channel of the flexible scope to clear the surgical scene. Additionally, or alternatively, a signal can be sent to a vacuum to suck out or remove particles through another channel of the flexible scope. Or, in another example, the signal can cause an illumination source such as an aiming beam to be activated or deactivated (e.g., turned on or off) based on the turbidity measurement or level.

At 710, the method 700 can optionally include obtaining a second signal from the one or more sensors and determining a second turbidity level at or near the distal tip of the endoscope. Determining the second turbidity level can be based at least in part on a signal from the one or more sensors and at least one of a correlation function or a lookup-table that correlates the signal from the one or more sensors to the level of turbidity. The determination made at 710 can be made subsequent or at a later time than the determination made at 706. The second turbidity level can thus represent a change in the turbidity level from the determination made at 706.

At 712, the method 700 can optionally include providing a second signal to adjust the at least one operating parameter (e.g., at least one of the suction or the irrigation at or near the distal tip of the flexible scope). The adjustment can be made based, at least in part on the second turbidity level (or the change in turbidity level) determined at 710. For example, if the second turbidity level is higher than the turbidity level determined at 706, then the amount of irrigation and/or suction can be increased. Conversely, if the second turbidity level is lower than the turbidity level determined at 706, the amount of irrigation and/or suction can be reduced or terminated completely. When the signal is used to control an illumination source, when the second turbidity level is different than the turbidity level determined at 706, the intensity or brightness of the illumination source such as the aiming beam can be adjusted (e.g., increased, decreased, or terminated) as desired or appropriate.

At 708 and/or 712, along with, separate from, or alternatively to the signal to control the irrigation and/or suction, a control signal can be sent to a lithotripsy emission source. For example, when the lithotripsy emission source is a laser, a control signal can be sent to increase, decrease, or terminate the amount of laser radiation being emitted based on the turbidity level, the second turbidity level, a change in the turbidity level, and/or amount of (or change in) surgical scene clarity.

FIG. 8 is a block diagram of an example of an apparatus, device, or machine 800 upon which any one or more of the techniques (e.g., methodologies) discussed herein may perform. In alternative embodiments, the machine 800 may operate as a standalone device or can be connected (e.g., networked) to other machines. The machine 800 can be connected to the medical device of FIG. 1, such as connected to the medical device via the I/O connection 108 and/or the accessory port 110. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations. Examples, as described herein, may include, or may operate by, logic or a number of components, or mechanisms. Circuit sets are a collection of circuits implemented in tangible entities that include hardware (e.g., simple circuits, gates, logic, etc.). Circuit set membership can be flexible over time and underlying hardware variability. Circuit sets include members that may, alone or in combination, perform specified operations when operating. In an example, hardware of the circuit set can be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components can be used in more than one member of more than one circuit set. For example, under operation, execution units can be used in a first circuit of a first circuit set at one point in time and reused by a second circuit in the first circuit set, or by a third circuit in a second circuit set at a different time.

Machine 800 (e.g., a computer system) may include a hardware processor 802 (e.g., a central processing unit (CPU), a graphics processing unit (GPU), a hardware processor core, field programmable gate array (FPGA), or any combination thereof), a main memory 804 and a static memory 806, some or all of which may communicate with each other via an interlink (e.g., bus) 830. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 can be a touch screen display. The machine 800 may additionally include a storage device 808 (e.g., drive unit), a signal generation device 818 (e.g., a speaker), a network interface device 820 connected to a network 826, and one or more sensors 816, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 808 may include a machine readable medium 822 on which is stored one or more sets of data structures or instructions 824 (e.g., software) embodying or used by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 808 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824. The term “machine readable medium” may include any non-transitory medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

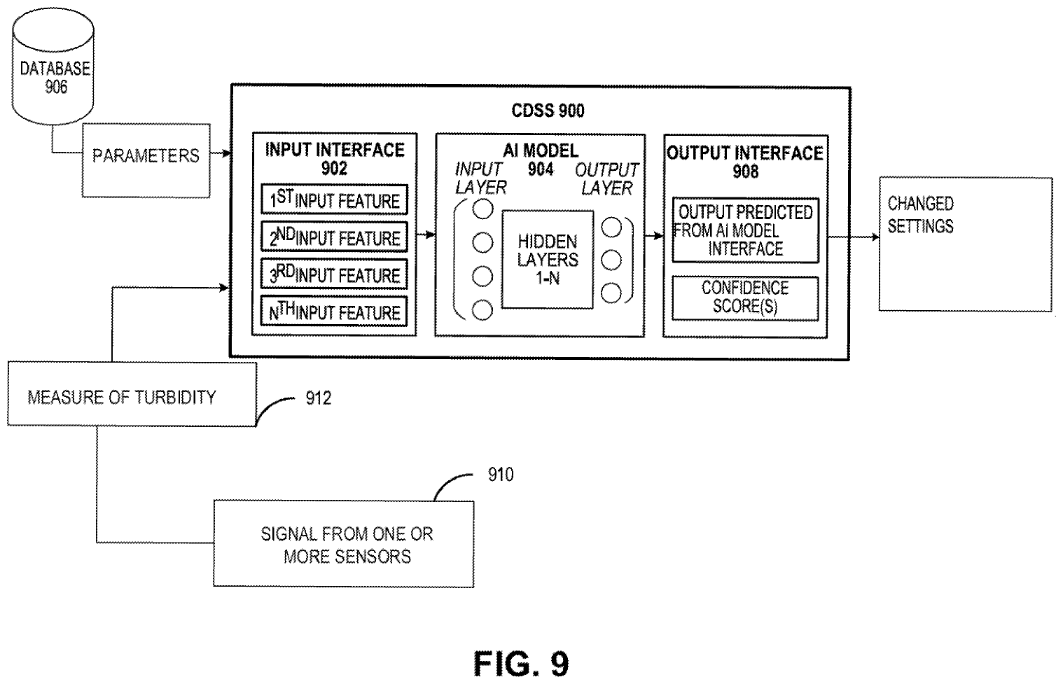

FIG. 9 illustrates a schematic diagram of an exemplary computer-based clinical decision support system (CDSS) 900 that can be configured to implement or recommended changed settings of an irrigation and/or suction system based on information about the turbidity of a surgical site during a medical procedure. In various embodiments, the CDSS 900 can include an input interface 902 through which information about a patient and/or the medical procedure the patient is scheduled for, which are specific to a patient, are provided as input features to an artificial intelligence (AI) model 904. The input features can also be provided to a processor, such as processor 802, which can perform an inference operation in which the information is applied to the AI model to generate the one or more changed laser settings, and a user interface (UI) through which one or more changed laser settings is communicated to a user, e.g., a clinician.

In some embodiments, the input interface 902 can be a direct data link between the CDSS 900 and one or more medical devices that generate at least some of the input features. For example, the input interface 902 may transmit information about the procedure and/or information from (e.g., a signal from) a sensor coupled to a medical device or scope directly to the CDSS 900 during a therapeutic and/or diagnostic medical procedure. Additionally, or alternatively, the input interface 902 can be a classical user interface that facilitates interaction between a user and the CDSS 900. For example, the input interface 902 may facilitate a user interface through which the user may manually enter information about the procedure that are specific to the patient. Additionally, or alternatively, the input interface 902 may provide the CDSS 900 with access to an electronic patient record from which one or more input features can be extracted. In any of these cases, the input interface 902 can be configured to collect one or more of the following input features in association with a specific patient on or before a time at which the CDSS 900 can be used to assess:
Information about the medical procedure;
Information about the medical device or devices to be used during the procedure;
Information about one or more settings of an irrigation system of or connected to the medical device or devices;
Information from one or more sensors 910, which can include a measure of conductance per centimeter of a solution at a surgical site, a degree of electrical conductivity of the solution, or a voltage and/or current measurement as discussed for FIG. 2, a voltage of the secondary coil discussed for FIG. 3, an amount of scattered light and/or the electrical signal discussed for FIG. 4, the amount of emitted light absorbed by the light sensor discussed for FIG. 5, and/or a particle count discussed for FIG. 1;
Information regarding a measure of turbidity 912 (e.g., derived from or based on the information from the one or more sensors);
Based on one or more of the above input features, the processor 802 can perform an inference operation using the AI model to generate a change in one or more irrigation settings to be implemented or a recommended change in one or more irrigation settings to be proposed to the user. For example, input interface 902 may deliver the information about the procedure, the devices, and/or the sensors into an input layer of the AI model which can propagate these input features through the AI model to an output layer. The AI model can provide a computer system the ability to perform tasks, without explicitly being programmed, by making inferences based on patterns found in the analysis of data. The AI model can explore the study and construction of algorithms (e.g., machine-learning algorithms) that may learn from existing data and make predictions about new data. Such algorithms can operate by building an AI model from example training data in order to make data-driven predictions or decisions expressed as outputs or assessments.

Two modes for machine learning (ML) include supervised ML and unsupervised ML. Supervised ML can use prior knowledge (e.g., examples that correlate inputs to outputs or outcomes) to learn the relationships between the inputs and the outputs. The goal of supervised ML includes to learn a function that, given some training data, best approximates the relationship between the training inputs and outputs so that the ML model can implement the same relationships when given inputs to generate the corresponding outputs. Unsupervised ML includes the training of an ML algorithm using information that is neither classified nor labeled and allowing the algorithm to act on that information without guidance.

Unsupervised ML can be useful in exploratory analysis because it can automatically identify structure in data.

Tasks for supervised ML include classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of supervised-ML algorithms include Logistic Regression (LR), Naive-Bayes, Random Forest (RF), neural networks (NN), deep neural networks (DNN), matrix factorization, and Support Vector Machines (SVM). Some tasks for unsupervised ML include clustering, representation learning, and density estimation. Some examples of unsupervised-ML algorithms include K-means clustering, principal component analysis, and autoencoders. Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data. In some examples, the AI model can be trained continuously or periodically prior to performance of the inference operation by the processor 802. Then, during the inference operation, the patient specific input features provided to the AI model can be propagated from an input layer, through one or more hidden layers, and ultimately to an output layer that corresponds to the changes to one or more of the laser settings. For example, when the turbidity of the surgical field is high, a change in the irrigation settings such the flow of irrigation fluid and/or an amount of suction can be propagated to the output layer. During and/or subsequent to the inference operation, the change in the irrigation settings can be communicated to the user via the user interface (UI) and/or automatically cause the processor 802 to automatically adjust the irrigation settings and proceed with the laser procedure using the new settings.

Example 1 is a medical system capable of determining at least one of turbidity or surgical scene clarity, the medical system comprising: one or more turbidity sensors coupled to a flexible scope; and processing circuitry, coupled to the one or more turbidity sensors, configured to: obtain a signal from the one or more turbidity sensors; determine a level of turbidity of a solution at or near a distal tip of the flexible scope; and based on the determined level of turbidity, provide a signal to control at least one of irrigation, suction, or lighting at or near the distal tip of the flexible scope.

In Example 2, the subject matter of Example 1 optionally includes wherein the processing circuitry is configured to determine the level of turbidity based at least in part on the signal from the one or more turbidity sensors and at least one of a correlation function or a lookup-table that correlates the signal from the one or more turbidity sensors to the level of turbidity.

In Example 3, the subject matter of any one or more of Examples 1-2 optionally include wherein the processing circuitry is configured to determine a level of scene clarity based at least in part on the signal from the one or more turbidity sensors and at least one of a correlation function or a lookup-table that correlates the signal from the one or more turbidity sensors to the level of scene clarity.

In Example 4, the subject matter of any one or more of Examples 1-3 optionally include wherein a first turbidity sensor of the one or more turbidity sensors is located inside a lumen or a working channel of the flexible scope.

In Example 5, the subject matter of Example 4 optionally includes wherein a second turbidity sensor of the one or more turbidity sensors is located external to the lumen or the working channel of the flexible scope.

In Example 6, the subject matter of any one or more of Examples 1-5 optionally include wherein the one or more turbidity sensors include: a first electrode; and a second electrode spaced laterally apart and offset from the second electrode; wherein at least one of the first electrode or the second electrode are connected to a sensing circuit, the sensing circuit comprising at least one of a voltage sensor or a current sensor configured to detect and measure at least one of a voltage signal or a current signal generated between the first electrode and the second electrode.

In Example 7, the subject matter of Example 6 optionally includes wherein at least one of the first electrode or the second electrode are located inside a lumen or working channel of the flexible scope.

In Example 8, the subject matter of any one or more of Examples 1-7 optionally include wherein the one or more turbidity sensors include: a first electromagnetic coil coupled to an alternating current (AC) source; and a second electromagnetic coil coupled to an AC voltmeter, the second electromagnetic coil located laterally spaced apart and offset from the first electromagnetic coil; wherein the AC source causes the first electromagnetic coil to induce an electric current that passes through a solution between the first electromagnetic coil and the second electromagnetic coil, wherein the electric current induces a voltage in the second electromagnetic coil to be measured by the AC voltmeter.

In Example 9, the subject matter of any one or more of Examples 1-8 optionally include a light source to emit light into a solution located at the distal tip of the flexible scope, and wherein the one or more turbidity sensors includes a light sensor configured to measure at least a portion the emitted light reflected back toward the distal tip of the flexible scope.

In Example 10, the subject matter of any one or more of Examples 1-9 optionally include a light source to emit light into a solution located at the distal tip of the flexible scope, and wherein the one or more turbidity sensors includes a light sensor located laterally spaced apart from the light source and configured to detect and measure a portion of the emitted light attenuated or absorbed by one or more solids suspended in the solution.

Example 11 is a medical system capable of determining at least one of turbidity or surgical scene clarity, the medical system comprising: a lithotripsy emission source; one or more turbidity sensors coupled to a flexible scope; and processing circuitry, coupled to the one or more turbidity sensors, configured to: obtain a signal from the one or more turbidity sensors; determine a level of turbidity of a solution proximate to a portion of the flexible scope; and based on the level of turbidity, provide a signal to control at least one of a lithotripsy emission source or at least one of irrigation, suction, or lighting at or near a distal tip of the flexible scope.

In Example 12, the subject matter of Example 11 optionally includes wherein the lithotripsy emission source includes a laser emission source, and wherein to control the lithotripsy emission source includes adjusting an intensity or an amount of radiation or laser light emitted from the laser emission source.

In Example 13, the subject matter of any one or more of Examples 11-12 optionally include wherein the lithotripsy emission source includes an acoustic vibration source, and wherein to control the lithotripsy emission source includes adjusting a frequency of an ultrasonic signal emitted by the acoustic vibration source.

In Example 14, the subject matter of any one or more of Examples 11-13 optionally include wherein the processing circuitry is further configured to: output a least one of an indication or a recommendation to a user on a graphical user interface (GUI) communicatively coupled to the medical system.

In Example 15, the subject matter of Example 14 optionally includes wherein the one or more turbidity sensors includes a particle counter located between the distal tip of the flexible scope and an irrigation port of a device connected to the flexible scope configured to determine an amount of material removed from a target during a lithotripsy procedure, and wherein the indication includes at least one of a turbidity percentage at or near the distal tip of the flexible scope, a scene clarity percentage at or near the distal tip of the flexible scope, a current ablation rate of the target, or an estimated amount of time required to complete ablation of the target.

Example 16 is a method of determining at least one of turbidity or surgical scene clarity, the method comprising: providing one or more turbidity sensors coupled to a flexible scope; obtaining a signal from the one or more turbidity sensors; determining a level of turbidity of a solution at or near a distal tip of the flexible scope; and providing, based on the level of turbidity, a signal to control at least one of ablation energy or at least one of irrigation, suction, or lighting at or near the distal tip of the flexible scope.

In Example 17, the subject matter of Example 16 optionally includes wherein determining the level of turbidity is based at least in part on the signal from the one or more turbidity sensors and at least one of a correlation function or a lookup-table that correlates the signal from the one or more turbidity sensors to the level of turbidity.

In Example 18, the subject matter of any one or more of Examples 16-17 optionally include determining a level of surgical scene clarity based at least in part on the level of turbidity.

In Example 19, the subject matter of Example 18 optionally includes wherein determining a level of scene clarity is based at least in part on the signal from the one or more turbidity sensors and at least one of a correlation function or a lookup-table that correlates the signal from the one or more turbidity sensors to the level of scene clarity.

In Example 20, the subject matter of any one or more of Examples 16-19 optionally include providing, based at least in part on the level of turbidity, a control signal to a lithotripsy emission source coupled to a medical device including the flexible scope.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A medical system capable of determining at least one of turbidity or surgical scene clarity, the medical system comprising:
one or more turbidity sensors coupled to a flexible scope; and
processing circuitry, coupled to the one or more turbidity sensors, configured to:
obtain a signal from the one or more turbidity sensors;
determine a level of turbidity of a solution at or near a distal tip of the flexible scope; and
based on the determined level of turbidity, provide a signal to control at least one of irrigation, suction, or lighting at or near the distal tip of the flexible scope.

2. The medical system of claim 1, wherein the processing circuitry is configured to determine the level of turbidity based at least in part on the signal from the one or more turbidity sensors and at least one of a correlation function or a lookup-table that correlates the signal from the one or more turbidity sensors to the level of turbidity.

3. The medical system of claim 1, wherein the processing circuitry is configured to determine a level of scene clarity based at least in part on the signal from the one or more turbidity sensors and at least one of a correlation function or a lookup-table that correlates the signal from the one or more turbidity sensors to the level of scene clarity.

4. The medical system of claim 1, wherein a first turbidity sensor of the one or more turbidity sensors is located inside a lumen or a working channel of the flexible scope.

5. The medical system of claim 4, wherein a second turbidity sensor of the one or more turbidity sensors is located external to the lumen or the working channel of the flexible scope.

6. The medical system of claim 1, wherein the one or more turbidity sensors include:
a first electrode; and
a second electrode spaced laterally apart and offset from the second electrode;
wherein at least one of the first electrode or the second electrode are connected to a sensing circuit, the sensing circuit comprising at least one of a voltage sensor or a current sensor configured to detect and measure at least one of a voltage signal or a current signal generated between the first electrode and the second electrode.

7. The medical system of claim 6, wherein at least one of the first electrode or the second electrode are located inside a lumen or working channel of the flexible scope.

8. The medical system of claim 1, wherein the one or more turbidity sensors include:
a first electromagnetic coil coupled to an alternating current (AC) source; and
a second electromagnetic coil coupled to an AC voltmeter, the second electromagnetic coil located laterally spaced apart and offset from the first electromagnetic coil;
wherein the AC source causes the first electromagnetic coil to induce an electric current that passes through a solution between the first electromagnetic coil and the second electromagnetic coil, wherein the electric current induces a voltage in the second electromagnetic coil to be measured by the AC voltmeter.

9. The medical system of claim 1, comprising:
a light source to emit light into a solution located at the distal tip of the flexible scope, and wherein the one or more turbidity sensors includes a light sensor configured to measure at least a portion the emitted light reflected back toward the distal tip of the flexible scope.

10. The medical system of claim 1, comprising:
a light source to emit light into a solution located at the distal tip of the flexible scope, and wherein the one or more turbidity sensors includes a light sensor located laterally spaced apart from the light source and configured to detect and measure a portion of the emitted light attenuated or absorbed by one or more solids suspended in the solution.

11. A medical system capable of determining at least one of turbidity or surgical scene clarity, the medical system comprising:
a lithotripsy emission source;
one or more turbidity sensors coupled to a flexible scope; and
processing circuitry, coupled to the one or more turbidity sensors, configured to:
obtain a signal from the one or more turbidity sensors;
determine a level of turbidity of a solution proximate to a portion of the flexible scope; and
based on the level of turbidity, provide a signal to control at least one of a lithotripsy emission source or at least one of irrigation, suction, or lighting at or near a distal tip of the flexible scope.

12. The medical system of claim 11, wherein the lithotripsy emission source includes a laser emission source, and wherein to control the lithotripsy emission source includes adjusting an intensity or an amount of radiation or laser light emitted from the laser emission source.

13. The medical system of claim 11, wherein the lithotripsy emission source includes an acoustic vibration source, and wherein to control the lithotripsy emission source includes adjusting a frequency of an ultrasonic signal emitted by the acoustic vibration source.

14. The medical system of claim 11, wherein the processing circuitry is further configured to:
output a least one of an indication or a recommendation to a user on a graphical user interface (GUI) communicatively coupled to the medical system.

15. The medical system of claim 14, comprising:
wherein the one or more turbidity sensors includes a particle counter located between the distal tip of the flexible scope and an irrigation port of a device connected to the flexible scope configured to determine an amount of material removed from a target during a lithotripsy procedure, and wherein the indication includes at least one of a turbidity percentage at or near the distal tip of the flexible scope, a scene clarity percentage at or near the distal tip of the flexible scope, a current ablation rate of the target, or an estimated amount of time required to complete ablation of the target.

16. A method of determining at least one of turbidity or surgical scene clarity, the method comprising:
providing one or more turbidity sensors coupled to a flexible scope;
obtaining a signal from the one or more turbidity sensors;
determining a level of turbidity of a solution at or near a distal tip of the flexible scope; and
providing, based on the level of turbidity, a signal to control at least one of ablation energy or at least one of irrigation, suction, or lighting at or near the distal tip of the flexible scope.

17. The method of claim 16, wherein determining the level of turbidity is based at least in part on the signal from the one or more turbidity sensors and at least one of a correlation function or a lookup-table that correlates the signal from the one or more turbidity sensors to the level of turbidity.

18. The method of claim 16, comprising:
determining a level of surgical scene clarity based at least in part on the level of turbidity.

19. The method of claim 18, wherein determining a level of scene clarity is based at least in part on the signal from the one or more turbidity sensors and at least one of a correlation function or a lookup-table that correlates the signal from the one or more turbidity sensors to the level of scene clarity.

20. The method of claim 16, comprising:
providing, based at least in part on the level of turbidity, a control signal to a lithotripsy emission source coupled to a medical device including the flexible scope.
